(19) United States
(12) Patent Application Publication
(10) Pub. No.: US 2026/0069792 A1
(43) Pub. Date: Mar. 12, 2026

(54) INJECTION DEVICE AND ADD-ON DEVICE

(71) Applicant: Sanofi, Paris (FR)

(72) Inventors: Stefan Alt, Frankfurt am Main (DE); Michael Helmer, Frankfurt am Main (DE)

(21) Appl. No.: 19/107,432

(22) PCT Filed: Aug. 28, 2023
(86) PCT No.: PCT/EP2023/073461

§ 371 Date: Feb. 27, 2025

(30) Foreign Application Priority Data
Aug. 30, 2022 (EP) 22315198.6

(51) Int. Cl. A61M 5/315; A61M 5/24; G06K 7/10

(52) U.S. Cl. CPC A61M 5/31568; A61M 5/2422; G06K 7/10366; A61M 2005/2485; A61M 2205/3592; A61M 2205/52; A61M 2205/581; A61M 2205/6054; A61M 2205/702

(57) ABSTRACT

Disclosed is an injection device for injecting of a dose of a medicament that includes a housing to accommodate a medicament container containing the medicament, a drive mechanism for injecting of the dose of the medicament from the medicament container, and a machine-readable identification comprising an electronic circuit with a memory configured to store at least one of a device information, a container information and a use-related data, wherein the memory is writable by an add-on device configured to detachably connect to the injection device.

### CROSS-REFERENCE TO RELATED APPLICATIONS

The present application is the national stage entry of International Patent Application No. PCT/EP2023/073461, filed on Aug. 28, 2023, and claims priority to European Application No. EP 22315198.6, filed on Aug. 30, 2022, the disclosures of which are incorporated herein by reference.

### TECHNICAL FIELD

The present disclosure relates to the field of injection devices for injecting a dose of a medicament and to add-on devices for such injection devices, in particular, to add-on devices attachable to a pen-type injector. In another aspect, the disclosure relates to an injection system comprising an injection device and an add-on device. In further aspects the disclosure relates to a method of monitoring operation of an injection device by making use of an add-on device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is beneficial to assist the user by making use of an external electronic device, such as a mobile electronic device, e.g. implemented as a smartphone, a tablet computer, or a smart watch. A software application provided on such external electronic devices may interact with the user and may provide instructions or recommendations to the user of how to correctly use the injection device.

In order to control and to supervise administering of medication conducted by users or patients themselves it is desirable to provide an automated detecting and logging of a repeated and regular use of the drug delivery device. A rather automated recording of doses injected by a user would offer a significant advantage over a manual dose logging in terms of security and convenience.

There exist numerous add-on devices configured for use with injection devices that offer an electronic detection and monitoring of repeated dose injection procedures.

Typically, such add-on devices or auxiliary devices can be detachably connected to an injection device. An add-on device typically comprises a detector or detector arrangement operable to detect a date and/or time when the user sets or injects a dose of the medicament. Some add-on devices also provide a quantitative measurement of a size of a dose currently set or dispensed. Some add-on devices are intended for use with a series of injection devices. This may particularly apply with disposable injection devices, that are intended to become discarded after use or after the medicament located therein has been used up.

Generally, there exists a variety of different injection devices, which may be equipped with different drugs or medicaments but are generally operable to be used with one and the same type of an add-on device. While it is common practice to characterize or to label injection devices and/or medicaments of different types and/or of different concentration by a clear and distinct label or identification the detachable connection with an add-on device may impose certain disadvantages with regard to an unintended mix-up of attaching an add-on device intended for use with a first injection device is erroneously attached to a second injection device.

Moreover, some scenarios of use may include an alternating detachment and re-attachment of an add-on device to at least two different injection devices, e.g. equipped with different drugs or medicaments. When detaching an add-on device from an injection device a digital logging of a dosing history or dose monitoring may be no longer possible and may have to be discarded.

It is therefore desirable to set aside such disadvantages and to provide an improved injection device and/or an improved add-on device as well as an improved method of supervising a monitoring operation of a single or of multiple injection devices.

### SUMMARY

In one aspect there is provided an injection device for injecting of a dose of a medicament. The injection device comprises a housing to accommodate a medicament container. The medicament container contains the medicament, e.g. a liquid injectable medicament. The injection device further comprises a drive mechanism operable to inject the dose of the medicament from the medicament container. The injection device further comprises a machine-readable identification comprising an electronic circuit with a memory. The memory is configured to store at least one of a device information, a container information and a user-related data. The memory is writable by an add-on device configured to detachably connect to a portion of the injection device.

Typically, the injection device is implemented as a handheld injection device. It may comprise a pen-type injector. The injection device may be implemented all-mechanically. It may be void of any electrical or electronic components. The one and only electric or electronic component may be the machine-readable identification of the injection device. The machine-readable identification may be implemented as an electronic identifier. The machine-readable identification may comprise a passive wireless communication tag or an active wireless communication tag, such as a passive or active RFID tag, NFC tag or UWB tag. Insofar the injection device may be void of any active electric or electronic components.

By providing a machine-readable identification on or inside an injection device, which memory is writable or modifiable by an add-on device configured to detachably connect to a portion of the injection device, particular device information can be provided to the add-on device, e.g. for conducting a correct calibration or initialization of the add-on device. Moreover, by way of a writable memory the add-on device is enabled to store use-related data in the memory of the electronic circuit of the machine-readable identification. Hence, use-related data may be stored directly on or inside the injection device.

With some examples the use-related data contains information about a previous use of the injection device. Use-related data may contain a dosing history of the injection device, being indicative of a time and/or a date at which a dose of a particular size has been set or injected by making use of the respective injection device. The process of dose setting or dose injecting has been monitored or is actually monitorable by the add-on device.

In response to monitor and/or or to detect a dose setting procedure or dose dispensing procedure the add-on device may store a respective dose setting or dose injecting information in a local storage and/or may be operable to write or store the respective dosing related data in the memory of the electronic circuit of the machine-readable identification. The machine-readable identification may be regularly written or overwritten by the add-on device. Hence, the add-on device may be configured to store any recorded or detected dosing event, dose setting and/or dose injecting and may record each of the such a dosing event immediately in the memory of the machine-readable identification. In this way, the machine-readable identification as provided on or inside the injection device is always up to date with regards to the dosing history of the injection device.

In case that the add-on device should be detached from the injection device the respective information, hence the use-related data can remain in the machine-readable identification and can be read and/or resumed when the injection device is reconnected or reattached to the same add-on device or to another add-on device. The respective add-on device, when reattached, is then operable to read-out the use-related data from the machine-readable identification and thus automatically obtains a dosing history of the injection device.

According to a further example the use-related data is indicative of a status of use of the injection device. The status of use is determinable by the add-on device. The status of use may contain information such as: total number of doses set or injected, a total amount of medicament injected by the injection device, a type, a name and/or concentration of medicament injected by the injection device, a LOT number of a medicament container and other medicament- or medicament container-related information that may be useful for the actual or future use of the injection device.

According to a further example the housing of the injection device comprises a body and a container part. The container part is detachably connectable to the body. The body accommodates the drive mechanism. The container part constitutes or contains the medicament container. In particular, the container part is sized and shaped to accommodate the medicament container. At least one of the body and the container part comprises the machine-readable identification, which, when provided on or in the body or drive mechanism comprises an electronic body identifier.

When provided on or in the container part the machine-readable identification comprises an electronic container identifier.

In effect and specifically when the injection device is implemented as a disposable injection device, i.e. intended to become discarded in its entirety when the medicament contained in the medicament container has been used up, it may be sufficient when the injection device comprises only one machine-readable identification. With other examples and specifically when the injection device is implemented as a reusable injection device the container part is detachably connected or connectable to the body. It may be then of particular advantage when at least one of the body and the drive mechanism comprises the electronic body identifier and when the container part or the medicament container comprises the electronic container identifier.

The container part may be provided as preassembly or subassembly of the injection device, wherein the preassembly or subassembly comprises the container part to accommodate the medicament container. Here, the container part serves as a secondary container sized and configured to accommodate the medicament container implemented as a primary medicament container. With some examples the container part comprises a cartridge holder and the medicament container comprises or constitutes a cartridge arranged inside the container part or cartridge holder. With some examples there may be provided a variety of container parts and/or a variety of medicament containers that are dedicated to be used with only one of a plurality of available bodies or drive mechanisms.

Insofar it is of particular benefit when the container part and the body comprise at least one machine-readable identification. Hence, the machine-readable identification provided in or on the container part comprises the electronic container identifier and the respective machine-readable identification provided on or inside the body of the injection device comprises the electronic body identifier. The electronic body identifier and the electronic container identifier may be separately read-out by the add-on device in order to electronically identify the respective container part or medicament container and to identify the respective body or drive mechanism.

By way of the electronic body identifier and the electronic container identifier different types of injection devices can be electronically labeled thus allowing to electronically distinguish between different types of container parts or medicament containers and body parts or drive mechanisms of a variety of injection devices.

The electronic body identifier as well as the electronic container identifier are both connectable with the add-on device configured for mechanical attachment to the injection device. Hence, the memory, e.g. in form of a digital storage of the respective electronic circuit of the electronic body identifier, and the electronic-container identifier can be read-out and/or written by the add-on device. This way, the add-on device may write or store the same or different information in the memories of the electronic body identifier and the electronic container identifier, respectively.

For instance, the add-on device may be operable to store use-related data in at least one or both of the memory of the electronic body identifier and the memory of the electronic container identifier. Moreover, the add-on device may be also operable to read data from one of the electronic body identifier and the electronic container identifier and to write the respective information into the memory of the other one of the electronic body identifier and the electronic container identifier. This way, an existing matching or a combination of a body or drive mechanism with a container part or a medicament container can be stored in the memory of at least one of the electronic body identifier and the electronic container identifier. This way not only an actual combination of one of a container part and a container with one of a drive mechanism and a body but also a history of such combinations can be electronically stored in the memory of at least one of the electronic body identifier and the electronic container identifier.

According to a further example the electronic body identifier contains the device information. The electronic container identifier contains the container information.

With some examples the container information is indicative of at least one of a type of a medicament contained in the medicament container a concentration of a pharmaceutical substance of the medicament contained in the container, a manufacturing date of the medicament, a use by date of the medicament or, a LOT number of the medicament or medicament container and/or environmental parameters, such as temperature, humidity or radiation intensity to which the medicament container has been exposed.

The container information may further include information about the type of the container in which the medicament is currently stored or contained. Insofar, the container information may contain container specific information, such as a type of a primary container, e.g. filled with the medicament or a type of a secondary container, e.g. a container part of an injection device, such as a cartridge holder configured to receive a primary container and configured for attachment to a body of a housing of an injection device.

With some examples the machine-readable identification is provided in an electric or electronic storage. It may be provided in a digital storage document such as a memory of an integrated circuit. Accordingly, the reader or transceiver on the electronic module comprises an electronic or digital reader operable to read the digital storage of the machine-readable identification. With some examples the machine-readable identification comprises one of an active or passive RFID, NFC or UWB tag readable by a respective RFID, NFC or UWB reader or transceiver of the electronic module.

With further examples the machine-readable identification comprises a magnetic code, readable by a respective magnetic reading or transceiving device of the electronic module.

The device information may comprise information or data with regard to the type of the injection device. The device information may be specific about a type of an injection mechanism or drive mechanism of the injection device. The device information may thus characterize the dose setting and/or dose dispensing operability of the respective injection device. The device identification may further contain data or information about suitable medicaments or medicament containers to be exclusively used with the respective injection device.

With some examples the drive mechanism comprises a piston rod, which in order to expel a predefined amount of a medicament has to move a predefined longitudinal displacement relative to the housing of the injection device. The drive mechanism is characterized by a particular feeding rate of its piston rod per unit of the medicament dose. The feeding rate of the drive mechanism has to match with the medicament and/or with a medicament container in use with the drive mechanism or injection mechanism.

By way of the reader or transceiver the add-on device is capable to obtain or to read the device information and/or the container information as provided on or inside the injection device. Typically, reading of the device information and/or the container information is enabled and becomes exclusively possible when the add-on device is attached to the injection device. Insofar and upon installing or mounting the add-on device to or on the injection device the reader or transceiver of the add-on device can be autonomously triggered to read or to obtain the machine-readable identification thereby obtaining at least one of the device information and the container information.

The device information and/or the container information can then be used to calibrate the dose size determination or dose size calculation to be conducted by the module processor of the electronic module of the add-on device.

According to a further example at least one of the electronic body identifier and the electronic container identifier contains the use-related data. The use-related data may be writable by the add-on device directly attachable or connectable to the injection device.

With other examples the use-related data as stored in at least one of the electronic body identifier and the electronic container identifier may be also provided and/or stored by a transceiver of an external electronic device, such as a smartphone, a smartwatch or a tablet computer.

The use-related data may be stored in one or in both of the electronic container identifier and the electronic body identifier. At least one of the electronic body identifier and the electronic container identifier comprises a memory with a memory block for the use-related data.

The use-related data may be indicative of the total amount of medicament extracted or dispensed by the injection device during use with the add-on device attached to the injection device. Use-related data may be further indicative of a dosing history of the injection device. It may contain data such as a point of time or data at which as dose dispensing action took place. It may further contain data about the amount of medicament dispensed with each dosing or injection action.

According to a further example the electronic circuit of the machine-readable identification comprises an antenna to wirelessly communicate with at least one of a transceiver of the add-on device and a device transceiver of an external electronic device. The antenna is connected to the memory of the electronic circuit of the machine-readable identification.

Typically, and with further examples the electronic circuit of the machine-readable identification also comprises a processor connected with the antenna and connected with the memory. The processor may be operable to manage reading and/or writing of data from the memory and in the memory. Moreover, the processor may manage transmission of wireless signals via the antenna of the electronic circuit. By way of an antenna the memory of the electronic circuit can be read or can be written wirelessly. This allows to attach or to arrange the machine-readable identification inside the injection device. This way, the machine-readable identification can be concealed inside the injection device and may further provide an anti-counterfeiting function for the injection device. It may be invisible from outside the injection device and may be only or exclusively readable or writable when a suitable add-on device or external electronic device is in a well-defined position or configuration relative to the antenna of the machine-readable identification.

According to a further example the machine-readable identification comprises a passive RFID transponder. With some examples the machine-readable identification may comprise an active RFID transponder. With some examples the memory of the machine-readable identification is a non-volatile memory. Hence, even in the absence of electric power or electric energy the data or information stored in the memory of the electronic circuit of the machine-readable identification remains. With some examples the machine-readable identification comprises a NFC tag or UWB tag. Implementation of an NFC tag has the benefit that reading or writing of information from and/or to the machine-readable identification is only possible when a respective reader or writing device, e.g. in form of the add-on device or an external electronic device, is in close vicinity and hence in a transmission range of the respective NFC transponder or NFC tag.

According to a further example the machine-readable identification comprises a pliable substrate attachable to a sidewall of at least one of the body, the container part and a barrel of the medicament container. The pliable substrate may be provided with a printed electronic circuit. Hence, the electronic circuit of the machine-readable identification may be printed on the pliable substrate. Also, the antenna of the machine-readable identification may be a printed conductive structure, which is printed on the pliable substrate. The pliable substrate may comprise a flexible foil or the like flexible substrate, which can be wrapped or otherwise attached to e.g. a tubular-shaped structure of at least one of the body, the container part and the barrel of the medicament container.

The pliable substrate of the machine-readable identification may be further provided with printed information being indicative of the respective type of the injection device, the container part or the medicament located inside the medicament container. Insofar, the machine-readable identification may be either integrated into a label attached to at least one of the body, the container part and the barrel of the medicament container. Furthermore, the machine-readable identification may provide or may constitute an electronic label attached or attachable to at least one of the body, the container part and the medicament container. With some examples the pliable substrate may be provided with an adhesive layer, providing and allowing an easy attachment of the machine-readable identification to at least one of the body, the container part and the barrel of the medicament container by way of an adhesive connection.

The drive mechanism comprises at least one movable component, which is subject to a movement relative to the housing or medicament container during at least one of setting of the dose and injecting of the dose of the medicament. With some examples the movable component is one of a dose dial being rotatable relative to the housing for setting of a dose, a dial sleeve rotatable relative to the housing during setting of the those or dispensing of the dose, a drive sleeve or a drive member, which is subject to a rotational and/or longitudinal motion relative to the housing during at least one of setting of the dose and dispensing of the dose. With some examples the movable component is a clicker or clicking element operable to generate an audible sound recordable by the sensor of the add-on device. With further examples the movable component is a piston rod operably engaged with a piston of the medicament container for displacing the piston relative to the medicament container during injecting of the dose. With further examples the movable component is a single dose indicating member, whose movement and/or position relative to the housing or device body is indicative of the size of a dose currently set. With further examples the movable component is a last dose member, whose position relative to the housing of the injection device is directly indicative of the residual amount of medicament left in the medicament container or cartridge.

With another aspect the present disclosure also relates to an add-on device for attaching to an injection device. The injection device is configured to inject a dose of a medicament. The injection device comprises a housing to accommodate a medicament container, wherein the medicament container contains the injectable medicament. The injection device further comprises a drive mechanism for injecting of the dose of the medicament from the medicament container. The injection device further comprises a machine-readable identification comprising an electronic circuit with a writable memory configured to store at least one of a device information, a container information and a use-related data.

Typically, the add-on device is configured for use with an injection device as described above. Insofar, all features, effects and benefits as described above in connection with the injection device equally apply to the add-on device; and vice versa. The add-on device further comprises a device body detachably fastenable to a portion of the injection device. With some examples the device body is fastenable to a body of the injection device. With further examples the device body is attachable or fastenable to e.g. a dose dial of the injection device, which is rotatable and/or longitudinally displaceable relative to the body for setting of the dose and/or for dispensing or injecting of the dose of the medicament. The add-on device further comprises an electronic module. The electronic module comprises a module processor and a transceiver. The transceiver and/or the module processor or are operable to read, to receive, to alter or to generate at least one of the device information, the container information and the use-related data. The electronic module is further operable to write and/or to store at least one of the device information, the container information and the use-related data in the memory of the electronic circuit of the machine-readable identification of the injection device.

The add-on device may be configured to monitor for repeated actions of use of the injection device. The add-on device may be configured to log or to monitor a repeated use of the injection device. Specifically, the add-on device, especially the electronic module is operable to detect and to record a date and/or a point of time at which a dose setting or dose dispensing action took place with the injection device. With further examples the add-on device is further configured to quantitatively measure a size of a dose currently set, dispensed or injected by the injection device when the add-on device is attached to the injection device.

Insofar, the add-on device is configured to record a dosing history of the injection device, wherein the dosing history contains information about the time, the date and the amount of medicament dispensed by the injection device.

Since the add-on device is capable to store at least one of the device information, the container information and the use-related data in the memory of the electronic circuit of the machine-readable identification, the injection device can be directly provided with respective use-related data obtained or generated by the add-on device. Typically, the add-on device may be also operable to read at least one of a device information, a container information and a use-related data from the machine-readable identification when attached to the injection device and to modify at least one of the device information, the container information and the use-related data and to store the correspondingly modified information in the memory of the machine-readable identification.

In this way and if for instance the injection device is provided with a first and a second machine-readable identification, e.g. with an electronic body identifier and an electronic container identifier the add-on device may be configured to read the data from the electronic body identifier and to store the respective data or a portion thereof in the memory of the electronic container identifier. Vice versa, the add-on device may be operable to read data from the electronic container identifier and to store the respective data or a portion thereof in the electronic body identifier of the injection device. This way, an electronic body identifier and an electronic container identifier, which are provided as separate and non-interconnected electronic identifiers on or inside the injection device can be logically connected.

Hence, the data or information stored in at least one of the electronic body identifier and the electronic container identifier may not only be indicative of generic data or information of this respective identifier but may also be provided with data or identification information of the other one of the electronic body identifier and the electronic container identifier. This way, a kind of a pairing history of mutual pairings or combinations of individual components of the injection device can be stored, e.g. a combination of at least one of a body and a drive mechanism of the injection device with at least one of a container part or medicament container with which the body or drive mechanism has been engaged or used.

According to a further example the add-on device comprises a sensor operable to quantitatively determine at least one of a position and a movement of the movable component relative to at least one of the housing, the first medicament container and the device body, typically when the device body is attached and/or fixed to the injection device. The sensor is operable to generate a respective sensor signal(s) in response to the detection of a position and/or of a movement or velocity of movement of the movable component of the injection device.

Furthermore and according to another example the module processor of the add-on device is operable to determine or to calculate a size of the dose of the medicament, which is set or dispensed by the injection device, on the basis of the sensor signal or on the basis of a sequence of sensor signals obtained during operation of a dose setting or dose injecting component of the injection device.

With some examples the module processor is further operable to determine or to calculate the size of the dose not only on the basis of the sensor signal but also on the basis of at least one of the container information and the device information as obtained from the machine-readable identification.

With the present add-on device, and by the above described acquisition of at least one of a device information and a container information a somewhat automated calibration of the dose size determination or dose size calculation can be provided. Hence, the add-on device itself is capable to derive and/or to process medicament container specific information and/or device specific information, e.g. in the course of assembly to the injection device. In response to obtain the container information and/or the device information the add-on device may be configured to conduct a calibration routine. This way the measurable sensor signals as obtained by the sensor of the add-on device can be translated or re-calculated into correct dose size information.

According to a further example the electronic module further comprises a module memory connected to the module processor. The module memory and/or the module processor is or are operable to store at least one of the device information and the container information as stored information and/or to store a dosing history, wherein the dosing history includes a number of dose sizes dispensed or injected by the injection device and determined or calculated by the module processor.

According to a further example the module processor is operable to store at least one of a size of an injected those, a number of injected doses and a time or date of dose injection as the use-related data in the memory of the machine-readable identification. This way, the machine-readable identification of the injection device can be directly provided with use-related data being indicative of the dosing history of the prior use of the injection device. In case that the injection device should be attached or reattached to another add-on device, the respective add-on device may read-out the respective use-related data and may resume or continue to record a dosing history of the respective injection device.

According to another example of the add-on device the module processor is operable to read use-related data from the memory of the machine-readable identification. The module processor is further operable to determine or to calculate a residual amount of the medicament contained in the medicament container on the basis of the use-related data.

Typically, the electronic container identifier as e.g. provided on a medicament container and/or as provided on or inside a container part of the injection device may be provided with stored information being indicative of a residual amount of medicament left inside the medicament container. Hence, information about an effective residual volume or residual amount information of the medicament in the medicament container can be directly stored in an electronic and hence digital memory of the machine-readable identification of the respective container part or medicament container. This way, the medicament container itself or the container part receiving or containing the medicament container is directly provided with relevant information about the actual state or status of the medicament container.

Storing of use-related data directly in an electronic container identifier allows to arbitrarily combine the medicament container or container part even with other drive mechanisms or bodies of other injection devices and/or to logically connect or pair the respective medicament container with different add-on devices. This way a rather flexible and universal way of using and/or combining the medicament container e.g. with a variety of suitable drive mechanisms or injection devices as well as with a variety of different add-on devices can be provided.

According to another aspect the present disclosure further relates to an injection system. The injection system comprises an injection device as described above and further comprises an add-on device as described above.

Specifically, the injection device of the injection system comprises a housing to accommodate a medicament container provided with the medicament. The injection device further comprises a drive mechanism for injecting of a dose of the medicament from the medicament container. The injection device further comprises a machine-readable identification comprising an electronic circuit with a writable memory configured to store at least one of a device information, a container information and a use-related data. The add-on device comprises a device body detachably fastenable to a portion of the injection device. The add-on device further comprises an electronic module comprising a module processor and a transceiver operable to read, to receive, to alter or to generate at least one of the device information, the container information and the use-related data.

The electronic module is operable to store at least one of the device information, the container information and the use-related data in the module memory. Since the injection system comprises an injection device and an add-on device as described above, all features, effects and benefits as described above with regards to any one of the injection device and the add-on device equally apply to the injection system; and vice versa.

According to a further aspect the present disclosure also relates to a method of monitoring operation of an injection device, wherein the injection device is configured for injecting of a dose of a medicament. The method comprises the steps of attaching an add-on device to a portion of the injection device. The injection device comprises a machine-readable identification comprising an electronic circuit with a writable memory. The writable memory is configured to store at least one of a device information, a container information and a use-related data. The method further comprises the step of reading, receiving, altering or generating at least one of the device information, the container information and the use-related data by an electronic module of the add-on device. Finally, at least one of the device information, the container information and the use-related data is stored in the memory by the electronic module.

With some examples it may be exclusively the electronic module of the add-on device that is configured to store at least one of the device information, the container information and a use-related data in the memory. With further examples it may be a transceiver of an external electronic device, such as a smartphone, a smartwatch and a tablet computer to store at least one of the device information, the container information and the use-related data in the memory of the machine-readable identification of the injection device.

The method of monitoring operation of the injection device may be conducted by making use of an injection system as described above and hence by making use of at least one of the injection device as described above and by making use of at least one of the add-on device as described above. Insofar, all features, effects and benefits as described above in connection with any one of the injection device, the add-on device and the injection system equally apply to the method of monitoring operation of the injection device; and vice versa.

According to a further example storing of at least one of the device information, the container information and the use-related data includes establishing of a wireless communication link between a transceiver of the electronic module and an antenna of the electronic circuit of the machine-readable identification. By way of a wireless communication link between the transceiver of the add-on device or a transceiver of an external electronic device and the antenna of the electronic circuit of the machine-readable identification there can be provided a wireless information exchange or data exchange between the machine-readable identification of the injection device and the add-on device or external electronic device. In this way the machine-readable identification can be arranged not only on the outside but also inside the injection device. It may be concealed by a housing or by any functional component of the injection device and may thus also provide an anti-counterfeiting feature.

According to another aspect the present disclosure relates to an add-on device for attaching to an injection device. The injection device comprises a housing, a medicament container and a drive mechanism. The drive mechanism is configured to inject a dose of the medicament from the medicament container. The housing comprises a body and a container part detachably connectable to the body. The body comprises an electronic body identifier containing device information and the container part comprises an electronic container identifier containing the container information. The add-on device comprises a device body detachably fastenable to a portion of the injection device and further comprises an electronic module comprising a module processor and a transceiver. The processor and/or the transceiver is/are operable to read the device information from the electronic body identifier and to read the container information from the electronic container identifier.

The electronic module may be operable to read the device information and the container information separately and/or sequentially. The electronic module may be operable to store the device information or container information in a module memory. Hence, the add-on device is capable or operable to separately identify the container part or medicament container and the body part or drive mechanism of an injection device. Insofar, the add-on device may keep track or may identify individual components of an injection device, which individual components may be detachable or replaceable for e.g. resetting or reconstituting the injection device.

According to a further example the electronic module is operable to compare the device information with the container information. The electronic module is further operable to determine, if the device information matches with the container information. Insofar the add-on device is operable and capable to individually identify the drive mechanism or body as well as to individually identify the container part and/or the medicament container. In response to a respective identification the electronic module is operable to compare the container information with predefined device information and vice versa to compare device information with predefined container information. Hence, the electronic module may be provided with a reference data or matching data sets being indicative, which kind of a medicament container or container part can be or should be used with which kind of a drive mechanism or body part of an injection device.

In this way, the add-on device is operable or configured to assert or to determine a suitable matching between a drive mechanism and a medicament container and hence a suitable or intended matching between a body and a container part of a reusable injection device.

According to a further example the electronic module is operable to activate a locking mechanism of the drive mechanism when the container information does not match the device information. The locking mechanism is operable to block at least one of setting of the dose and dispensing of the dose. For this, the add-on device and/or its electronic module may comprise a locking controller that is electrically connected or electromechanically connectable or engageable with the locking mechanism of the drive mechanism. In case that the add-on device determines that the electronic container identifier is indicative of a particular container part or medicament container that does not match the drive mechanism or body of an injection device as retrieved from the electronic body identifier, the locking mechanism can be activated.

Vice versa it is also conceivable or possible with the add-on device that the locking mechanism of the drive mechanism of the injection device is in a locking mode per default and that for unlocking of the locking mechanism of the drive mechanism it is required to connect the add-on device to the injection device. It may be then only or exclusively upon a mutual matching of device information obtained from the body identifier and container information obtained from the electronic container identifier that the locking controller of the add-on device unlocks the locking mechanism of the drive mechanism of the injection device. In this way setting and/or dispensing of a dose of a medicament is only allowed when: a) the add-on device is connected to the injection device and b) when the first the drive mechanism matches with the medicament container or medicament located inside the medicament container. This way, misuse of the injection device can be effectively prevented thus leading to an enhancement of patient safety especially in the field of home medication or self-treatment making use of the injection device.

According to a further example the electronic module is operable to generate a user perceptible alert signal when the container information does not match the device information, which container information has been retrieved or obtained from the container identifier and which device information has been retrieved or obtained from the electronic body identifier. Generation of a user perceptible alert signal may be generated by the add-on device itself. Here, a signal generator of the add-on device may be activated.

A user-perceptible alert may be also triggered by the add-on device, e.g. by way of a signal transmission between the add-on device and at least one external electronic device. Here, an alert signal may be wirelessly transmitted from a transceiver of the electronic module to a respective transceiver of the external electronic device, which when receiving the respective alert signal is operable to generate a respective user perceptible alert, which may be of audible, visual or palpable type.

According to a further aspect the present disclosure also relates to a method of inspecting an injection device. The injection device comprises a housing, a medicament container and a drive mechanism. The drive mechanism is configured to inject a dose of the medicament from the medicament container. The housing of the injection device comprises a body and a container part, wherein the container part is detachably connectable to the body. The body comprises an electronic body identifier containing device information and the container part comprises an electronic container identifier containing container information. The method comprises the steps of reading the device information from the electronic body identifier by an electronic module of an add-on device configured for attaching to the injection device and reading the container information from the electronic container identifier by the electronic module. Thereafter and in a further step the device information is compared with the container information and a degree of matching between the device information and the container information is determined. Determination of a degree of matching between the device information and the container information may be conducted on the basis of a predefined or given matching schedule which defines an allowable pairing between different medicament containers with different drive mechanisms.

According to a further aspect the present disclosure also relates to a computer program, e.g. a computer program product comprising computer readable instructions, which when executed by a module processor of an add-on device as described above cause the module processor to conduct the steps of the method as described above.

Typically, the computer program is executable by an add-on device as described above. It is further operable to conduct a method of monitoring operation of the injection device with the add-on device as described above. Insofar, the computer program and its computer readable instructions are operable to cause a processor of the add-on device to conduct a method of monitoring or checking the injection device as described above. The computer readable instructions may be executable by a processor of an add-on device as described above.

Additionally, or alternatively and when an external electronic device of an injection system as described above is configured to read-out the machine-readable identification of the injection device the computer program and its computer readable instructions may be at least in part executable by a processor of the external electronic device.

The computer program is to be executed by a processor of an add-on device as described above in order to monitor and/or record operation of the injection device when the add-on device is attached thereto. Insofar, all features, effects and benefits as described above in connection with the add-on device and/or described in connection with the method of configuring the add-on device as well as any effects, features and benefits as described above in connection with the injection system equally apply to the computer program; and vice versa. The present disclosure further discloses and proposes a computer program including computer-executable instructions for performing the method according to the disclosed method/device/system in one or more of the examples enclosed herein when the program is executed on a processor, computer or computer network. Specifically, the computer program may be stored on a computer-readable data carrier. Thus, specifically, one, more than one or even all of the method steps as indicated above may be performed by using a computer or a computer network, typically by using a computer program.

The present disclosure further discloses and proposes a computer program product having program code means, in order to perform the method according to the disclosed method/system in one or more of the embodiments enclosed herein when the program is executed on a computer or computer network. Specifically, the program code means may be stored on a computer-readable data carrier.

Further, the present disclosure discloses and proposes a data carrier having a data structure stored thereon, which, after loading into a processor, computer or computer network, such as into a working memory or main memory of the processor, computer or computer network, may execute the method according to one or more of the examples disclosed herein.

The present disclosure further proposes and discloses a computer program product with program code means stored on a machine-readable carrier, in order to perform the method or parts thereof according to one or more of the examples disclosed herein, when the program is executed on a processor, computer or computer network. As used herein, a computer program product refers to the program as a tradable product. The product may generally exist in an arbitrary format, such as in a paper format, or on a computer-readable data carrier. Specifically, the computer program product may be distributed over a data network.

With a further example the injection device comprises a dose dial and a trigger, e.g. provided at a proximal longitudinal end of the injection device. The injection device may be implemented as a pen-type injector. The dose dial and/or the trigger may be provided at a proximal longitudinal end of the pen-type injector. Here, the machine-readable identification may be provided in or on the dose dial and/or the trigger. The add-on device may be configured for releasable or detachable fastening to the proximal end of the injection device.

The add-on device, in particular its device body may comprise a receptacle to fit onto the dose dial and/or trigger. By implementing or arranging the machine-readable identification in or on the trigger or dose dial a geometric distance between the machine-readable identification and a reader or transceiver of the add-on device for readout of the machine-readable identification can be reduced to a minimum thus allowing to implement the transceiver or reader of the add-on device as a wireless near field transceiver operable to read a respective near field communication tag of the machine-readable identification of the device. This way, readout of the machine-readable identifier can only take place when the add-on device is correctly mounted to the proximal end of the injection device.

According to a further example the injection system comprises an external electronic device, e.g. implemented as a smartphone, a smart watch or as a tablet computer. The external electronic device may be operable to conduct at least one or several steps of the method of monitoring operation of the injection device.

Generally, the scope of the present disclosure is defined by the content of the claims. The disclosure is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des (B30) human insulin, Lys(B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1: 2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates an example of an injection device,

FIG. 2 shows the process of assembling an add-on device to a proximal end of the injection device,

FIG. 3 shows an injection device in a longitudinal schematic cross-section,

FIG. 4 shows a configuration of an injection system including the injection device, the add-on device and at least one external electronic device,

FIG. 5 shows a pairing between the external electronic device and the add-on device,

FIG. 6 shows a visual illustration on a display of an external electronic device to assist a user in setting up the injection device,

FIG. 7 shows a further step of reading the electronic container identifier provided on the container part of the injection device

FIG. 8 shows a visual feedback as provided on the external electronic device in response to a read-out of information from the electronic container identifier,

FIG. 9 shows the injection device and the add-on device before finally assembling the add-on device to the injection device,

FIG. 10 shows a visual feedback provided on a display of an external electronic device after detection of a correct pairing of components of the injection device,

FIG. 11 shows a further example of an add-on device when attached to the injection device, FIG. 12 shows the example of FIG. 11 before attaching the add-on device to the injection device,

FIG. 13 shows a variably connection of the add-on device to one of two available injection devices,

FIG. 14 shows a block diagram of the add-on device, the injection device and the external electronic device,

FIG. 15 shows a block diagram of the electronic circuit of the machine-readable identification,

FIG. 16 shows an example of the electronic circuit on a substrate,

FIG. 17 shows a flowchart of a method of configuring the add-on device.

### DETAILED DESCRIPTION

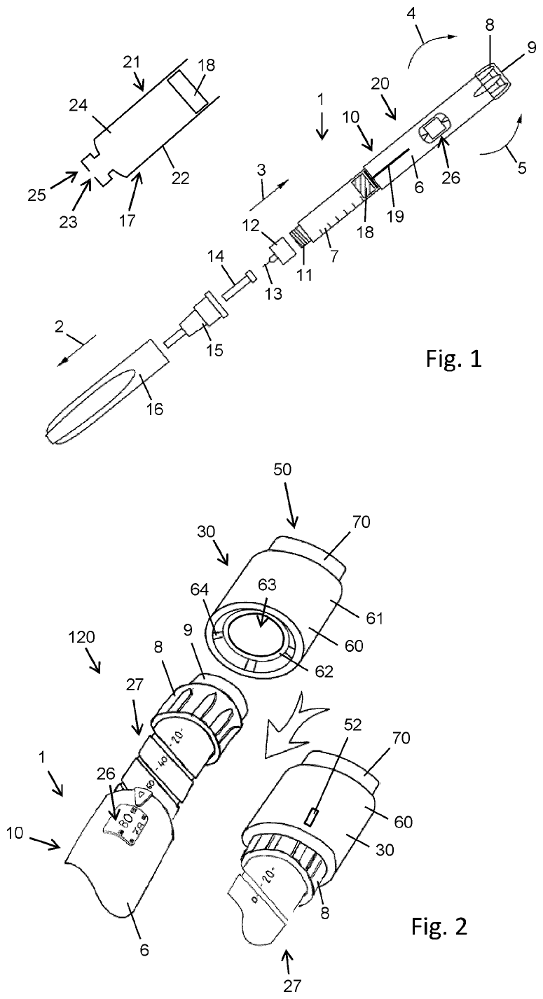

FIG. 1 shows an example of a drug delivery device, which is implemented as a handheld injection device 1. The injection device 1 may comprise or may be implemented as a pen-type injector. It may be implemented as a disposable injection device or as a reusable injection device. With some examples the injection device 1 is implemented as an autoinjector. The injection device 1 is of elongated shape. It may extend along a longitudinal direction. Towards a longitudinal distal direction 2 the drug delivery device 1 comprises a dispensing end for dispensing or injecting the medicament 24. Towards the proximal direction 3 the injection device 1 comprises at least one of a dose member 8 and a trigger 9, by way of which a dose of equal or individual or different size can be set and dispensed, respectively.

The injection device 1 comprises a housing 10. The housing 10 may comprise numerous housing components, such as a body 6 and a container part 7, e.g. implemented as a cartridge holder 7. The body 6 may be sized and configured to accommodate a drive mechanism 20. The container part 7 is sized and configured to accommodate a medicament container 21, e.g. implemented as a cartridge containing the liquid medicament 24. The medicament container 21 comprises a tubular-shaped barrel 22 sealed towards the distal end by a seal 23. The seal 23 may comprise a pierceable septum fixed to an outlet 25 of the medicament container 21. Towards a proximal end the interior of the barrel 22 is sealed by a piston 18 or stopper, which is slidably disposed inside the barrel 22.

By advancing the piston 18 towards the distal direction 2 a dose of the medicament 24 can be expelled from the medicament container 21. In use the medicament container 21 is arranged inside the container part 7. The drive mechanism 20 of the injection device 1 comprises a piston rod 19, which is displaceable in distal direction 2 for advancing the piston 18 towards the outlet 25 of the medicament container 21. Details of the drive mechanism are not further illustrated and described here. With some examples, the drive mechanism 20 may be implemented as an all-mechanical drive mechanism, where a user has to provide an entirety of a dispensing force required to move the piston rod 19 and hence the piston 18 in distal direction 2. With other examples, the drive mechanism comprises a mechanical energy storage configured to provide at least a portion of the dispensing force. Examples of drive mechanisms can be found e.g. in WO2004/078241 A1, WO 2014/033197 A1 or WO 2014/033195 A1 the entirety of which are herein incorporated by reference.

With some examples, as e.g. described or shown in FIG. 2 the injection device 1 and hence the drive mechanism 20 may comprise a dial extension 27, which projects and moves in proximal direction 3 from a proximal end of the body 6 when or during setting a dose and which returns into its initial distal end position during a dose injection procedure. For this, a user may use a thumb 114 of his hand 110 to exert a distally directed pressure onto the trigger 9 thereby urging the dial extension 27 in distal direction 2 during and/or for a dose injection procedure.

For setting or dialing of a dose a user may twist or rotate the dose dial 8, e.g. in a dose incrementing direction 4, hence in a clockwise sense as seen from the proximal end. For correcting a dose previously set the user may also rotate the dose dial 8 in an opposite dose decrementing direction 5. The size of the dose is typically illustrated in a window 26 provided in or on the body 6 of the injection device 1. Prior to inject a dose of the medicament 24 the distal end of the container part 7 or cartridge holder has to be connected with a needle assembly 12. For this, the distal end of the cartridge holder 7 comprises a connector 11, e.g. in form of a threaded interface to engage with a complementary shaped threaded counter interface of the needle assembly 12.

The needle assembly 12 is detachably or releasably fixable to the container part 7. It comprises a double-tipped injection needle 13. A proximal end of the injection needle (not shown) is configured to enter into a through opening at the distal end face of the connector 11 or container part 7 so as to pierce or to penetrate the seal 23 of the medicament container 21. The distal end of the injection needle 13 is typically covered by a detachable inner needle cap 14. The entirety of the needle assembly 12 may be covered by a detachable outer needle cap 15.

The container part 7 and hence a portion of the housing 10 is to be received in a protective cap 16, which is detachably connectable to the cartridge holder 7 or body 6.

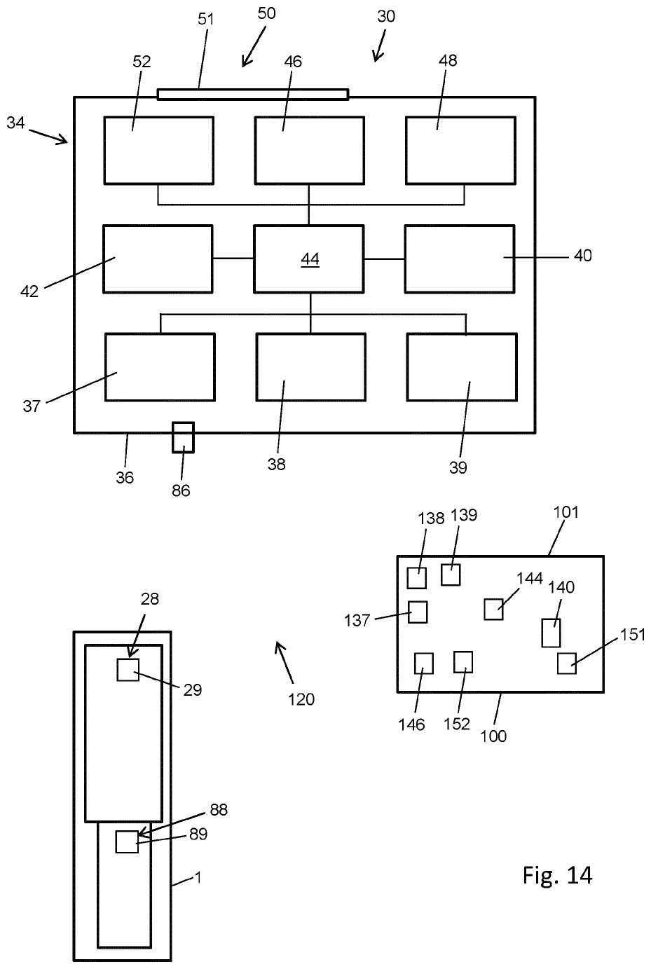

In FIGS. 2 and 4 there is shown an example of an add-on device 30 configured for fastening to the proximal end of the injection device 1. The add-on device 30 comprises a sensor 48 or sensor assembly, which is operable to detect, to recognize, to characterize and/or to measure an operation of the injection device 1. Typically, the sensor 48 of the add-on device 30 and as indicated in the block diagram of FIG. 14 is capable or operable to quantitatively determine or to measure at least one of a position and a movement of a movable component 80, 81, 82, 83 of the drive mechanism 20 of the injection device 1. The movable component 80, 81, 82, 83 is movable relative to at least one of the body 6 and the medicament container 21 during or for setting and/or injecting of a dose of the medicament 24.

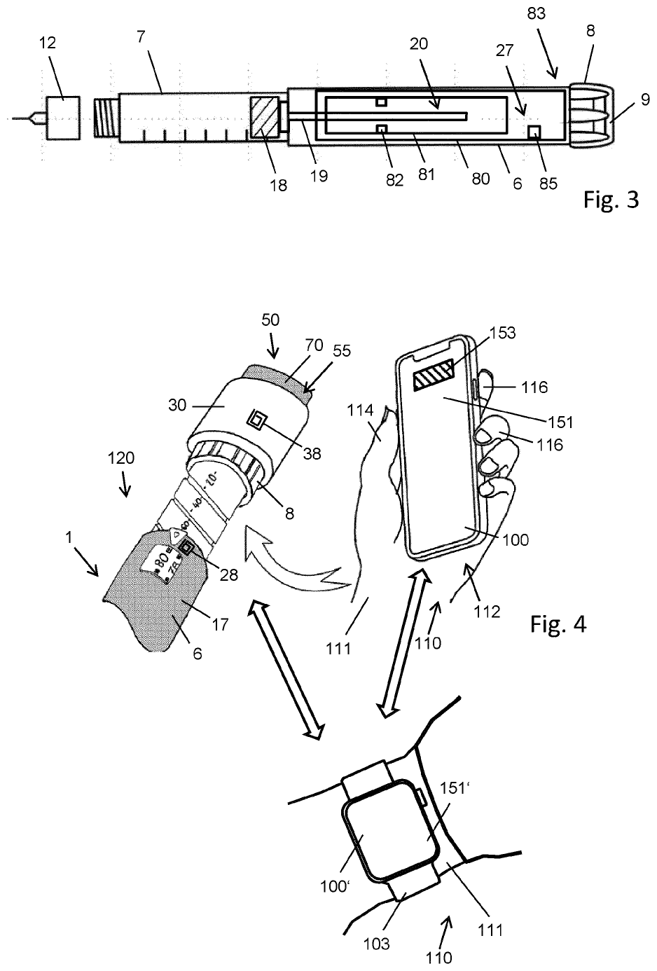

This movement or position is quantitatively measurable by the sensor 48. Typically, the sensor 48 is implemented as an electronic sensor. It is capable to detect or to measure a degree of longitudinal translation and/or rotation of the at least one movable component 80, 81, 82, 83 relative to the housing 10 or relative to the device body 60 when the add-on device 30 is attached to the injection device 1. Examples of movable components 80 are schematically illustrated in the cross-section of the injection device 1 according to FIG. 3

The sensor 48 of the add-on device 30 is capable or operable to generate a sensor signal or a sequence of sensor signals, which due to the coupling or connection to the module processor 44 can be processed by the module processor 44 in order to derive or to calculate a size of a dose currently set or dispensed.

With some examples the movable component 80 is a dose setting member or a dose setting sleeve. With further examples the movable component 81 is implemented as a drive member or drive sleeve 81 of the drive mechanism 20. With further examples the movable component is implemented as a volume indicator 82, e.g. as a single dose indicating member, whose position or configuration relative to the body 6 is directly indicative of the size of a dose currently set. With further examples the movable component 82, hence the volume indicator is a last dose indicating member, the position or configuration of which relative to the body 6 is indicative of a remaining amount of medicament left in the cartridge or medicament container 21.

With some examples the movable component 83 is or comprises an encoding, such as a digital encoding, which is movable relative to the sensor 48 such that the sensor is capable to count a number of discrete code portions of the encoding so as to gather respective quantitative movement information of the movable component 80. Generally, there may be provided different types of spatial encodings, such as an optical or visual encoding, an electrical or electrostatic encoding a magnetic encoding or mechanical encoding. Depending on the type of an encoding provided on or inside the movable component 80, 81, 82, 83 the sensor 48 is correspondingly configured. Hence, the sensor 48 may comprise at least one of an optical sensor, an electrical or electrostatic sensor and a magnetic sensor or mechanical sensor, such as a micromechanical switch, each of which being operable to quantitatively measure a relative position or relative movement of the movable component 80, 81, 82, 83.

With some examples the movable component 80, 81, 82, 83 is subject to a longitudinal and/or rotational movement relative to the body 6, relative to the medicament container 21 and/or relative to the device body 60 during at least one of setting of the dose and dispensing of the dose. Typically, the encoding as provided on or with the movable component 80, 81, 82, 83 is encoded along the direction of movement of the movable component relative to the sensor 48 so as to enable a respective quantitative measurement of the respective dose setting or dose dispensing movement.

As further indicated in FIG. 3 the drive mechanism 20 may be optionally equipped with a locking mechanism 85, which is operable by a locking controller of the add-on device 30 when the add-on device 30 is correctly assembled to the injection device 1. The locking mechanism 85 is operable to block or to lock the drive mechanism 20 so as to prevent unauthorized or unintended setting of a dose and/or dispensing or injecting of the dose. The locking mechanism 85 is controllable by the locking controller 86 of the add-on device 30. The locking mechanism 85 may be implemented electromechanically and may be electrically or electronically controlled by the locking controller 86 of the add-on device 30. This way, the add-on device 30 may block an unauthorized or unintended use of the injection device 1.

The add-on devices 30 as illustrated in the various FIGS. 2-14 is detachably connectable to the dose dial 8. It comprises a device body 60 with a tubular-shaped sidewall 61. Towards the distal end the sidewall 61 confines a receptacle 63, which is sized to receive the dose dial 8 and the trigger 9 of the injection device 1. For this, the inside of the sidewall 60 may comprise one or numerous fastening ribs 64, which are configured to provide a slip free fastening of the add-on device 30 to the dose dial 8.

The device body 60 comprises the receptacle 63 at a distal end section of the add-on device 30. The receptacle 63 is open towards the distal direction 2. It is sized and configured to fit onto the proximal end of the injection device 1. An inside surface of the sidewall 61 confining the receptacle 63 comprises numerous fastening ribs 64, e.g. of an elastic or elastically deformable material, such as an elastomeric material.

The fastening ribs 64 provide a friction fit with the dose dial 8 when the device body 60 is assembled to the dose dial 8 of the injection device 1. The receptacle 63 may be confined in longitudinal direction by a flange portion 62 protruding radially inwardly from the tubular-shaped sidewall 61 of the receptacle 63. The flange portion 62 is located proximally offset from the insert opening of the receptacle 63. The flange portion 62 may be of annular shape and may be configured to axially abut on a stepped down section at the proximal end of the dose dial 8. Hence, the trigger 9, which comprises a reduced diameter compared to the dose dial 8 may protrude in proximal direction 3 through the flange portion 62 while the flange portion 62 may rest or abut against the proximal end face of the dose dial 8 when the add-on device 30 is suitably fastened to the dose dial 8.

The add-on device 30 further comprises a movable part 70 protruding in proximal direction from the device body 60. The movable part 70 comprises or forms an auxiliary trigger or trigger button, which is configured to mechanically engage with the trigger 9 of the injection device 1 when the add-on device 30 is correctly assembled to the injection device 1.

Typically, the add-on device 30, in particular, the device body 60 is frictionally engageable with the dose dial 8. In this way, a user may apply a dose setting torque onto the dose dial via the device body 60. Instead of rotating the dose dial 8 for setting of a dose the user may simply rotate the device body 60 relative to the body 6 of the injection device 1. This way, the dial extension 27 may become subject to a dose incrementing dialing or rotating motion. A respective size of a dose currently set will then be displayed in the window 26 of the body 6 of the injection device 1 as illustrated in FIG. 2.

For dispensing of a dose the user has to depress the movable part 70, which may then be subject to a distally directed motion relative to the device body 60. The movable part 70, which may be in direct or indirect mechanical engagement with the trigger 9, may then apply a respective dispensing force onto the trigger 9 thereby initiating a dose dispensing action of the injection device 1.

The add-on device 30 as schematically illustrated in FIG. 14 comprises an electronic module 34. The electronic module 34 comprises a printed circuit board 36. The electronic module 34 comprises a module processor 44, an electronic and hence digital module memory 40 and a clock 42. Furthermore, the electronic module 34 comprises a power source 46 and the sensor 48. The electronic module 34 also comprises a signal generator 52 coupled to the processor 44 and/or coupled to the power source 46. The electronic module 34 further comprises at least one of a reader 37 and a wireless transceiver 38, 39. The add-on device 30 may also comprise a user perceptible device identification 50.

With some examples the device identification 50 comprises or includes the signal generator 52 coupled to the module processor 44 and operable or reconfigurable by the module processor 44. With some examples the device identification 50 may be indicative of the injection device 1 to which the add-on device 30 is actually connected or coupled to.

With some examples the signal generator 52 is operable to generate at least one of a visual, an acoustic and a haptic signal or device identification and is hence operable to produce or to generate a visual identifier, an acoustic identifier or a haptic identifier or a respective alert signal. With some examples the add-on device 30 comprises numerous signal generators 52 of equal or different type, e.g. optical, acoustic or haptic type.

With the example of FIG. 2 the signal generator 52 may comprise a speaker operable to generate an acoustic signal, wherein the acoustic signal may represent or constitute an alert signal e.g. in form of an acoustic code or sequence being characteristic for a specific configuration or setting of the injection device 1 or add-on device 30.

FIG. 4 is illustrative of an injection system 120 comprising at least the add-on device 30 and the injection device 1. Optionally, the injection system 120 comprises one or several external electronic devices 100, 101′. The external electronic device 100 is implemented as a smart phone comprising a housing 101 and being equipped with a device display 151 to visually illustrate various types of information, such as various illustrations 153 or notifications 154 to a user. The display 151 may be implemented as a touch sensitive display. The device display 151 may be operable to provide or to emulate a device signal generator 152. The device display 151 is operable to provide at least one of an illustration 153 and a notification 154 to a user.

The further optional external electronic device 100′ is implemented as a smartwatch comprising a respective housing and a display 151′ and further comprising a wristband 103 for fixing the external electronic device 100′ to the hand 110 or wrist 111 of a user. Like the external electronic device 100 also the external electronic device 100′ is operable to provide at least one of an illustration 153 and a notification 154 to the user. Generally, the external electronic device 100, 100′ may be equipped with a separate device signal generator 152, such as a speaker or a vibration module by way of which a user perceivable signal can be generated so as to provide an alert signal to a user of the respective device

The electronic device 100 is typically implemented as a handheld electronic device. It can be held in a hand 110 of a user or may be worn on the wrist 111 of a user. With the example as illustrated in FIG. 4 the electronic device 100 is held in a palm 112 of the user. The device display 151 is implemented as a touch sensitive display and can be operated by a thumb 114 and/or by various fingers 116 of the user's hand 110.

The electronic module 34 of the add-on device 30 as schematically illustrated in FIG. 14 comprises at least one of a reader 37 and a transceiver 38, 39 operable for wireless communication with at least one of a machine-readable identification 28, 88 of the injection device 1 or with a complementary or correspondingly implemented device reader 137 and/or device transceiver 138, 139 of the external electronic device 100, 100′.

With typical examples the transceivers 38, 138 are implemented as radiofrequency transceivers of near field type. The transceivers 38, 138 may be implemented as wireless short range transceivers or near field transceivers. They may be implemented as a so-called NFC transceivers allowing for wireless communication within a limited spatial range of a few centimeters or decimeters. The transceivers 38, 138 may be implemented as active and/or passive NFC tags or readers. Typically, the transceiver 38 is implemented as a passive NFC tag and the transceiver 138 is implemented as an active NFC tag or NFC reader.

The transceivers 39, 139 may be implemented as wireless local range transceivers, such as RFID, Bluetooth, Bluetooth low energy (BLE) or UWB transceivers. Also here, the transceiver 39 may be implemented as a passive communication tag whereas the transceiver 139 may be implemented as an active transceiver, hence as a reader. Typically, the transmission range of the transceivers 39, 139 is larger than the transmission range of the transceivers 38, 138. The transmission range of the transceivers 39, 139 may be in a range of several meters or decameters.

The optional reader 37, 137 may be implemented as an optical reader or optical information gathering unit, e.g. comprising an imaging system so as to capture or to record a visual or optical machine-readable identification 28, 88 as provided on or inside the injection device 1.

The external electronic device 100, 100′ typically comprises a device processor 144 coupled to an electronic device memory 140. The electronic device 100 further comprises a device power source 146. Also, the electronic device 100 comprises a device display 151, typically implemented as a touch sensitive display. The device display 151 may be operable to provide at least one of an illustration 153 and a notification 154 to a user.

The device transceiver 138 is operable to communicate with the transceiver 38 of the add-on device. The transceiver 139 is configured to communicate with the transceiver 39 of the add-on device 30. The reader 137 may be operable to read a visual identifier as provided on the injection device 1. With some examples the device reader 137 comprises an imaging system. It may comprise a camera objective and a spatially resolving detector in order to read or to capture a visual code, e.g. provided on an outside surface of the injection device 1 or as provided on an outside surface of the medicament container 21.

The external device 100 is configured to set up a communication link, e.g. a wireless communication link with the add-on device 30. For this, the local range transceiver 39 of the add-on device 30 may communicate wirelessly with the local range device transceiver 139 of the add-on device 100. Once a communication link has been established between the electronic device 100 and the add-on device 30 the electronic device 100 may be operable to visibly illustrate information or data of the injection device or medicament container 21 on the device display 151

The injection device 1 is provided with a machine-readable identification 28, 88 as schematically illustrated in FIG. 14. The machine-readable identification 28 comprises an electronic identifier 29. The machine-readable identification 88 comprises an electronic identifier 89. The electronic identifier 28 may comprise a passive wireless communication tag as schematically illustrated in FIG. 12.

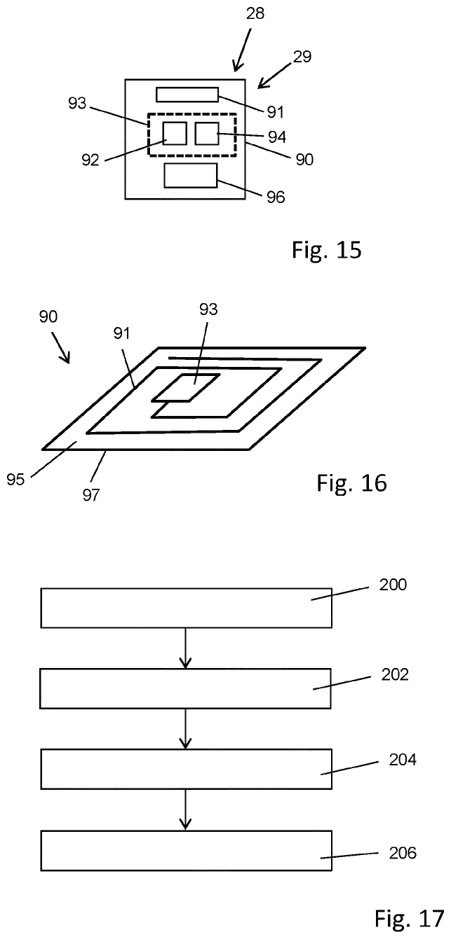

As illustrated in FIG. 15 the electronic identifier 29 may comprise an electronic circuit 90 featuring a wireless communication antenna 91 and an integrated circuit 93. The integrated circuit 93 comprises an electronic and hence digital memory 92 to and a processor 94 connected to the memory 92. Of course, the integrated circuit 93 and hence the processor 94 is connected to the antenna 91. The electronic identifier 29 may optionally comprise a power source 96 in order to provide electrical power or energy for the processor 94. The electronic circuit 90 may be provided on a planar substrate 95. The substrate 95 may be pliable or foldable. It may comprise a flexible structure or foil that allows for an easy mounting, fastening e.g. adhesive fastening, of the substrate 94 and hence of the entire electronic identifier 29 to at least one of the tubular shaped body 6, the container part 7 or the medicament container 21.

The electronic circuit 90 may be a printed electronic circuit, which is printed on the substrate 95. Here, the substrate 95 may comprise or constitute an e.g. flexible printed circuit board. The substrate 95 may be further provided with an adhesive layer 97 that allows for an easy and straightforward adhesive attachment of the machine-readable identification 28, 88 to a predefined portion on or inside the injection device 1. The electronic identifier 89 may comprise an identical or similar structure as the electronic identifier 29 as shown in FIGS. 12 and 13.

With some examples the machine-readable identification 28, 88 is implemented in a label 17 located on one of the body 6, the container part 7 or the medicament container 21. The label 17 comprises or contains the machine-readable identification 28, 88. With some examples the label 17 comprises a passive radiofrequency tag. With further examples the label 17 comprises machine-readable identifications 28, 88 implemented as a visual or optical code that can be captured by a reader 37 or device reader 137, which is implemented as or comprises an imaging system.

At least one of the reader 37, the device reader 137 or at least one of the transceivers 38, 39 or device transceivers 138, 139 is or are operable to read the machine-readable identification 28, 88 thereby obtaining at least one of a device information and a container information e.g. stored in the memory 92 of the electronic circuit 90.

Typically, the machine-readable identification 28 is provided or located on or inside the body 6 of the housing 10 of the injection device 1. The further machine-readable identification 88 is provided on or inside the container part 7. It may be provided on or inside the medicament container 21. With some examples the machine-readable identification 28 may be provided in or on the piston 18 of the medicament container 21.

The machine-readable identification 28, 88 may be implemented as a passive RF communication tag, such as a NFC tag. The information, e.g. the device information and/or the container information stored in at least one of the machine-readable identification 28, 88 is readable by the electronic module 34 and hence by any one of the transceivers 38, 39 of the add-on device 30 and/or by any of the transceivers 138, 139 of the external electronic device 100, 100′.

With some examples and especially when the injection device 1 is implemented as a disposable injection device, which does not support replacement of the medicament container 21 it is sufficient when the injection device 1 comprises only one machine-readable identification 28. It may be of particular advantage when the machine-readable identification 28 is provided at or near a proximal end 68 of the body 6. It may be provided on or inside the dose dial 8 or the trigger 9 so as to be within the transmission range of the transceiver 38, 39 when the add-on device 30 is attached to the proximal end of the injection device 1.

With some examples the distal end 67 of the body 6 is detachably connectable to a proximal end of the container part 7. Then, the injection device 1 is implemented as a reusable device that allows a replacement of the container part 7 and/or a replacement of the medicament container 21 inside the container part 7. With reusable injection devices 1 it may be of particular benefit when both, the body 6 and the container part 7 are provided with a separate machine-readable identification 28, 88. As illustrated in FIG. 14, the container part 7 is provided with the machine-readable identification 88 whereas the body 6 is provided with the machine-readable identification 28.

As illustrated in FIG. 9 the transceiver 38 may be located inside the receptacle 63 of the device body 60 so as to minimize a spatial distance to the machine-readable identification 28 as provided at or near the proximal end 68 of the body 6. By making use of near field communication technology for the machine-readable a notification 28, 88 and the respective transceivers 38, 138 it can be ensured, that the machine-readable identification and the device information and/or container information stored therein can only be acquired or obtained when the add-on device 30 and/or the external electronic device 100, 100′ is within the near field transmission range.

As further illustrated in FIG. 12, the receptacle 63 of the device body comprises an alignment feature 65 in form of a longitudinal recess or groove on the inside of the sidewall of the receptacle 63. Correspondingly, the proximal end of the dose dial 8 comprises a complementary shaped counter alignment feature 66, e.g. in form of a protrusion sized to fit into the recess or groove of the alignment feature 65. In this way, there can be provided a rotation inhibiting fastening for the add-on device 30 when attached to the housing 10 or dose dial 8 of the injection device 1.

When the alignment feature 65 is in mechanical engagement with the counter alignment feature 66 as shown in FIG. 11 there is provided an anti-rotation lock for the mutual fastening of the add-on device 30 to the injection device 1. In this way there can be guaranteed, that the identification 28 and hence the electronic identifier 29 always correctly aligns with the associated wireless transceiver 38 of the add-on device 30. There is hence provided a unique and well-defined mounting position for mounting the add-on device 30 to the injection device 1. When reaching the mounting position or mounting configuration as predefined by alignment feature 65 engaging the counter alignment feature 66 it is also guaranteed, that the machine-readable identification 28 and the transceiver 38 are at a minimum distance to each other. This allows for a somewhat undisturbed signal transmission between the identification 28 or electronic identifier 29 and the transceiver 38.

Generally, the external electronic device 100 may permanently or regularly synchronize with the add-on device e.g. by local range communication interfaces as provided by the transceivers 39, 139. Insofar, any information gathered or read by the add-on device 30 can be instantly transmitted to and further processed, e.g. visualized, by the external electronic device 100, 100′.

The injection device 1 is equipped with at least one machine-readable identification 28, 88 as illustrated in FIG. 14. The machine-readable identification 28 comprises an electronic identifier 29, denoted as electronic body identifier. The machine-readable identification 88 comprises a respective electronic container identifier 89. The respective electronic identifiers each comprise an antenna 91 and an integrated circuit 93 as described above in connection with FIG. 15.

With some examples the machine-readable identifications 28, 88 are implemented as a passive or active RFID and are operable to store at least one of a device information and a container information.

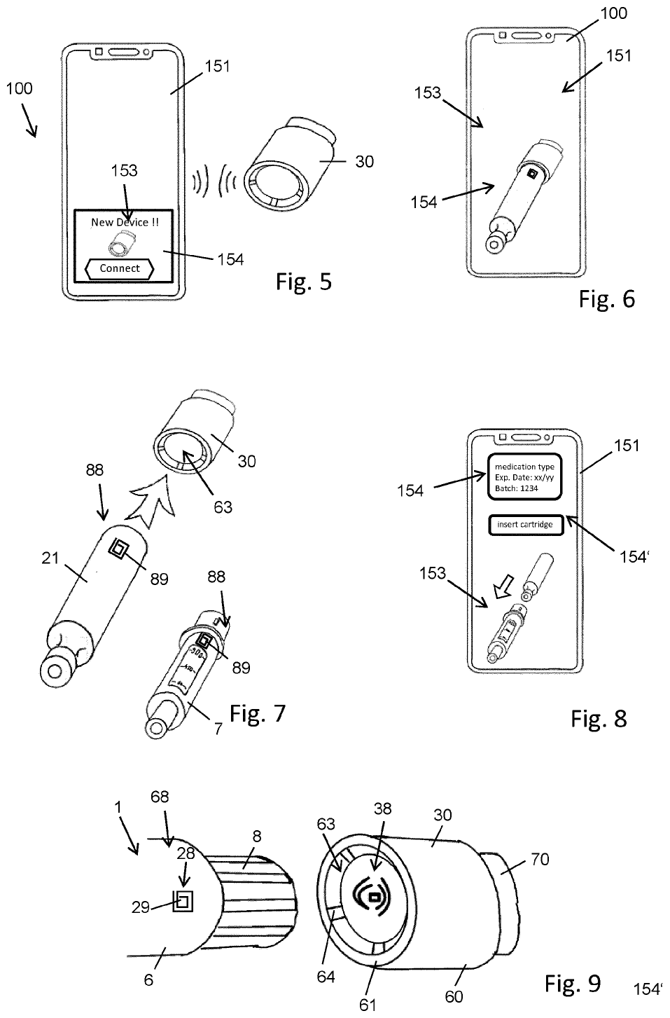

With the illustrated examples of FIGS. 5-13 it is of particular benefit when individual components of the injection device 1 are separately provided with a separate machine-readable identifications 28, 88. Here, the injection device 1 is implemented as a reusable injection device. The container part 7 is detachably connectable to the body part 6. While the container part 7 accommodates a medicament container 21 the body 6 contains or accommodates the drive mechanism 20. Each one of the container part 7 and the body 6 is provided with an own machine-readable identification 28, 88.

For instance, the container part 7, e.g. implemented as a cartridge holder, is provided with the electronic container identifier 89. The body 6 is provided with an electronic body identifier 29. As specifically illustrated in FIG. 7 the container identifier 89 may be either provided on or inside the container part 7 of the injection device 1. With some examples the electronic container identifier 89 may be provided on or inside a barrel 22 of the medicament container 21. With some examples the electronic container identifier 89 is provided on or inside the stopper or piston 18 of the medicament container 21.

The electronic container identifier 89 may comprise a memory 92 provided with container or medicament specific information. The electronic body identifier 29, in particular the memory 92 thereof is provided with information or data being specific of the body 6 and/or of drive mechanism 20. A user intending to use the injection device 1 in combination with the add-on device 30 may optionally pair or synchronize the add-on device 30 with an external electronic device 100 as illustrated in FIG. 5. Here, there may be set up a communication link between the device transceiver 139 of the external electronic device 100 and the transceiver 39 of the add-on device 30, respectively. The add-on device 30 may be provided with an add-on device-specific identifier that can be transmitted to the external electronic device 100. Upon receipt of a respective identification through the wireless communication link between the external electronic device 100 and the add-on device 30 the external electronic device may illustrate or otherwise audibly or haptically indicate a successful pairing with the add-on device 30. Accordingly, and when establishing a communication link between the add-on device 30 and the external electronic device 100 there may show up at least one of a visual illustration 153 and a respective notification 154 on the display 151 of the external electronic device 100.

Thereafter and as illustrated in FIG. 6 the external electronic device 100 and specifically an injection device specific app installed on the external electronic device 100 may prompt the user to use the add-on device 30 to read a machine-readable identification 88 as provided on one of the medicament container 21 and the container part 7. For this, the display 151 of the external electronic device 100 may provide a notification 154 and/or a respective illustration 153 and may be thus operable to assist or guide a user of the device(s) to bring at least one of the medicament container 21 and the container part 7 provided with such a medicament container 21 in close vicinity, hence in the transmission range of the transceiver 38 of the add-on device as illustrated in FIG. 6 or 7.

By bringing the electronic container identifier 89 in close vicinity to the transceiver 38 of the add-on device 30, the add-on device 30 is capable to read out the stored container information as stored in the memory 92 of the electronic container identifier 89. The respective information may be then illustrated on the display 151 as a notification 154 or illustration 153 being indicative of at least one of the medicament type, the expiration date, the batch number or LOT number with regard to a medicament production.

The display 151 may be provided with a further notification 154′ by way of which the user is prompted to execute further steps for setting up the injection device or injection system, 120. Here, the user may be prompted to insert the cartridge or medicament container 21 into the container part 7.

In other cases, wherein the medicament container 21 is e.g. preassembled inside the container part 70 may be sufficient to bring the container part equipped with the medicament container 21 in reading vicinity or within the transmission range of the add-on device 30 in order to enable a respective read-out of the container information.

Thereafter, the user may be prompted by the add-on device 100 to connect the add-on device 30 to the injection device 1. Here, the user may be prompted to connect the add-on device 30 with its receptacle 63 to the dose dial 8 as provided at or near the proximal end of the injection device 1. Then, and as illustrated in FIG. 9 the electronic body identifier 29 is provided at or near a proximal end 68 of the body 6. Alternatively, the electronic body identifier 29 may be provided on or inside the dose dial 8 so as to minimize a reading or transmission distance between the transceiver 38 of the add-on device 30 and the respective antenna 91 of the electronic circuit 90 of the electronic body identifier 29. When the add-on device 30 is suitably mounted on the injection device 1, the electronic body identifier 29 is within the transmission range of the transceiver 38 and the transceiver 38 is operable to read the device information from the electronic body identifier 29.

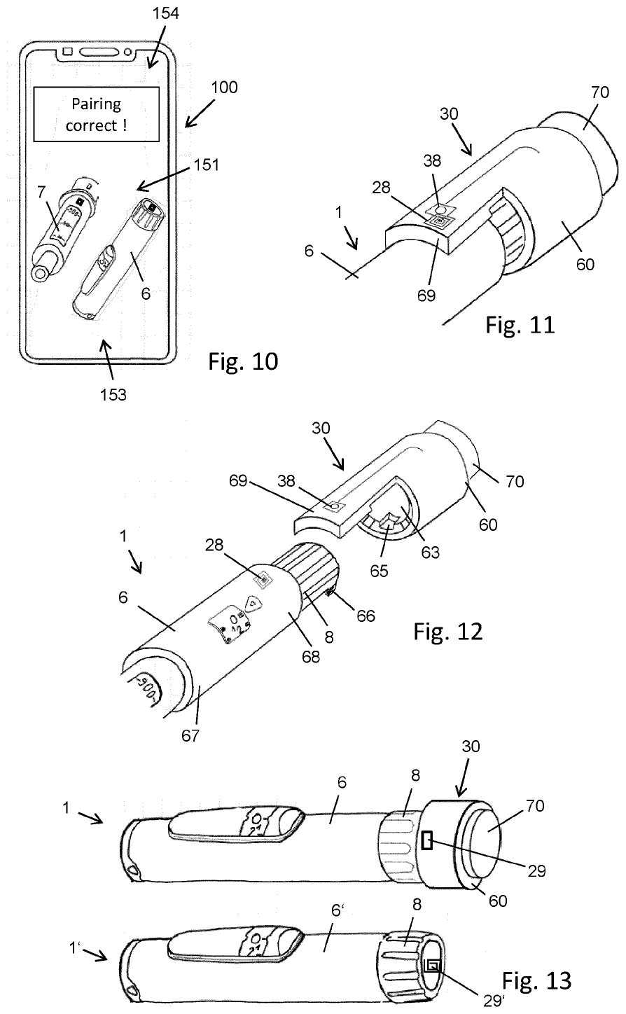

As further indicated in FIG. 10 the respective container information and the device information are mutually compared by at least one of the add-on device 30 and the external electronic device 100 in order to determine if the medicament container 21 matches with the drive mechanism 20 of the injection device 1 and/or to determine if the container part 7 matches with the body 6 or drive mechanism 20. The comparison of the container information and the device information may be conducted or executed by at least one of the module processor 44 and the device processor 144.

For this, the electronic module 34 may be operable to read the container information, the device information and/or the use-related data and may be further operable to forward and/or to transmit the respective information to the external electronic device 100, e.g. via a wireless local range communication link, which communication link is e.g. set up between the transceiver 39 and the device transceiver 139. This way, the computational power or digital data processing resources required for comparing of the acquired data can be exclusively provided by the external electronic device 100, thus allowing to simplify the computational demands of the add-on device 30 and to reduce the manufacturing costs of the add-on device 30.

With further examples a comparison may be provided by a third-party provider, e.g. by a database of a healthcare provider, which receives the respective container information and device information through a communication with the external electronic device 100 and wherein the external electronic device 100 receives a confirmation or disapproval with regard to a matching between injection device components that are provided with the electronic container identifier 89 and the electronic device identifier 29, respectively.

In case that the container information matches with the device information the external electronic device 100 may be configured to indicate to a user the correct pairing of the body 6 and the container part 7 as illustrated in FIG. 10. Both, the body 6 and the container part 7 can be individually identified by way of their respective electronic body identifier 29 and electronic container identifier 89. The matching of the medicament container 21 and/or the container part 7 with the body 6 may be visually illustrated on the display 151 of the external electronic device 100, e.g. in form of an illustration 153 of the recognized or identified components and/or by a respective notification 154 indicating to a user that the individually recognized or identified components of the injection device 1 mutually match.

In case of a successful matching the respective matching information may be also stored in the memory 92 of at least one of the electronic container identifier 89 and the electronic body identifier 29. This way, the respective components of the injection device 1 can be electronically marked or electronically linked to each other. Each individual component of the injection device 1 provided with a machine-readable identification 28, 88 can be provided with a history of use, which is written in the memory 92 by way of the add-on device 30 and/or by way of the external electronic device 100.

In case that the injection device 1 should be disassembled, e.g. for replacing an empty medicament container 21 the respective user-related data, e.g. data recorded, generated or modified during use of the injection device 1 and stored in a memory 92 of at least one of the electronic container identifier 89 and the electronic body identifier 29 can be read by the add-on device 30 and/or by the external electronic device 100 in order to determine if a particular body 6 is correctly reattached to the same or to another or different container part 7. In situations, wherein a container part 7 provided with an electronic container identifier 89 is mixed up with another container part during replacement of a medicament container such a mix-up can be electronically detected by acquisition, reading and comparing of the user-related data previously stored in the memory 92 of the respective electronic container identifier 89.

The add-on device 30 may be further equipped with a locking controller 86 operably engageable with a locking mechanism 85 of the drive mechanism 20. In case that the add-on device 30 and/or the electronic module 34 should detect a non-matching configuration of a body 6 with a container part 7 or medicament container 21 the electronic module 34, hence the module processor 44, may be operable to activate the locking mechanism 85 through the locking controller 86. With other scenarios it may be conceivable that the locking mechanism 85 is active per default and that unlocking of the locking mechanism 85 through the locking controller 86 is exclusively provided when:
a) the add-on device 30 is suitably mounted on the injection device 1 and/or
b) when the add-on device 30 detects a correct and suitable pairing of the body 6 or drive mechanism 20 with an intended medicament container 21 and/or container part 7.

With the further illustration of FIG. 13 it is indicated that the add-on device 30 can be used with a variety of different injection devices 1, 1′. Each injection device 1, 1′ is provided with an electronic body identifier 29, 29′. In the illustration of FIG. 13 the add-on device 30 is attached to the first injection device 1. In particular, the add-on device 30 is attached to the dose dial 8 of the first injection device 1. The add-on device 30 can be likewise mounted to a different, hence a second injection device 1′ provided with a different or with an identical body 6′.

The first injection device 1 is provided with a first electronic body identifier 29. The second injection device is provided with a second electronic body identifier 29′. Any operation of the injection device 1 can be suitably monitored and/or quantitatively measured by the add-on device 30 and the respective action or procedure can be stored in the memory 92 of the respective electronic body identifier 29, 29′.

This allows to detach the add-on device 30 from the first injection device 1 and to assemble or to re-attach the add-on device 30 to the second injection device 1′. Here, and upon attaching the add-on device 30 to the second injection device 1′, the information contained in the memory 92 of the second electronic body identifier 29 is read and a dose recording or dose history recording may be resumed.

In FIG. 17 a flowchart of executing a method of monitoring operation of the injection device 1 it is schematically illustrated. In a first step 200 that add-on device 30 is attached to a portion of the injection device 1. The add-on device 30 may be attached and/or fixed to the proximal end of the injection device 1. In a subsequent step 202 at least one of the device information and the container information is read by the wireless communication between the add-on device 30 and at least one of the electronic body identifier 29 and the electronic container identifier 89. Thereafter, and in an optional step 204 setting of a dose and/or injecting of a dose of a medicament is detected or measured by the sensor 48 of the add-on device. Here, the add-on device 30 is capable to record the dosing event in a module memory 40.

In the further step 206 the add-on device 30 stories or modifies data in the memory 92 of the machine-readable identification 28, 88 of the injection device. Here, use-related data, such as data being indicative of the dosing history or other device-relevant or container relevant data may be stored in any of the memories of the electronic body identifier 29 and/or of the electronic container identifier 89. Likewise, data previously retrieved from the electronic body identifier can be stored in the memory of the electronic container identifier and vice versa, data retrieved from the electronic container identifier can be stored in the electronic body identifier. This way, the individual components of the injection device, namely the body 6 or drive mechanism 20 can be linked to a particular medicament container 21 or container part 7.

### REFERENCE NUMBERS

1 injection device
2 distal direction
3 proximal direction
4 dose incrementing direction
5 dose decrementing direction
6 body
7 container part
8 dose dial
9 trigger
10 housing
11 connector
12 needle assembly
13 injection needle
14 inner needle cap
15 outer needle cap
16 protective cap
17 label
18 piston
19 piston rod
20 drive mechanism
21 medicament container
22 barrel
23 seal
24 medicament
25 outlet
26 window
27 dial extension
28 identification
29 electronic device identifier
30 add-on device
34 electronic module
36 printed circuit board
37 reader
38 transceiver
39 transceiver
40 module memory
42 clock
44 module processor
46 power source
48 sensor
50 indicator
51 display
52 signal generator
53 display section
54 display section
60 device body
61 sidewall
62 flange portion
63 receptacle
64 fastening rib
65 alignment feature
66 counter alignment feature
70 movable part
67 distal end
68 proximal end
69 extension
80 dose setting element
81 drive member
82 volume indicator
83 encoding
85 locking mechanism
86 locking controller
88 identification
89 electronic container identifier
90 electronic circuit
91 antenna
92 memory
93 integrated circuit
94 processor
95 substrate
96 power source
97 adhesive layer
100 electronic device
101 housing
103 wristband
110 hand
111 wrist
112 palm
114 thumb
116 finger
120 injection system
137 device reader
138 device transceiver
139 device transceiver
140 device memory
144 device processor
146 device power source
151 device display
152 device signal generator
153 illustration
154 notification

1.-17. (canceled)

18. An injection device for injecting of a dose of a medicament, the injection device comprising:
a housing to accommodate a medicament container containing the medicament,
a drive mechanism for injecting of the dose of the medicament from the medicament container, and
a machine-readable identification comprising an electronic circuit with a memory configured to store at least one of a device information, a container information and a use-related data, wherein the memory is writable by an add-on device configured to detachably connect to a portion of the injection device.

19. The injection device according to claim 18, wherein the use-related data is indicative of a status of use of the injection device, which status of use is determinable by the add-on device.

20. The injection device according to claim 18, wherein the housing comprises a body and a container part detachably connectable to the body, wherein the body accommodates the drive mechanism and wherein the container part constitutes or contains the medicament container and wherein at least one of the body and the container part comprises the machine-readable identification, which, when provided on or in the body or drive mechanism comprises an electronic body identifier and when provided on or in the container part comprises an electronic container identifier.

21. The injection device according to claim 20, wherein the electronic body identifier comprises the device information and wherein the electronic container identifier comprises the container information.

22. The injection device according to claim 20, wherein at least one of the electronic body identifier and the electronic container identifier comprises the use-related data.

23. The injection device according to claim 18, wherein the electronic circuit of the machine-readable identification comprises an antenna to wirelessly communicate with at least one of a transceiver of the add-on device and a device transceiver of an external electronic device.

24. The injection device according to claim 18, wherein the machine-readable identification comprises a passive RFID transponder.

25. The injection device according to claim 18, wherein the drive mechanism comprises at least one movable component, which is subject to a movement relative to the housing or relative to the medicament container during at least one of setting of the dose and injecting of the dose of the medicament and wherein at least one of a position and a movement of the movable component relative to at least one of the housing and the medicament container is measurable by a sensor of the add-on device when attached to the injection device.

26. An add-on device for attaching to an injection device, wherein the injection device is configured to inject a dose of a medicament and wherein the injection device comprises:
a housing to accommodate a medicament container containing the medicament,
a drive mechanism for injecting of the dose of the medicament from the medicament container, and
a machine-readable identification comprising an electronic circuit with a writable memory configured to store at least one of a device information, a container information and a use-related data, and
wherein the add-on device comprises:
a device body detachably fastenable to the injection device,
an electronic module comprising a module processor and a transceiver operable to read, to receive, to alter or to generate at least one of the device information, the container information and the use-related data and wherein electronic module is operable to store at least one of the device information, the container information and the use-related data in the memory.

27. The add-on device according to claim 26, further comprising a sensor operable to quantitatively determine at least one of a position and a movement of a movable component of the drive mechanism relative to at least one of the housing, the medicament container and the device body when the add-on device is attached to the injection device and wherein the sensor is operable to generate a sensor signal being indicative of at least one of the position and the movement of the movable component.

28. The add-on device according to claim 27, wherein the module processor is operable to determine or to calculate a size of the dose of the medicament, which is set or dispensed by the injection device, on the basis of the sensor signal.

29. The add-on device according to claim 28, wherein the module processor is further operable to store at least one of a size of an injected dose, a number of injected doses and a time or date of dose injection as the use-related data in the memory.

30. The add-on device according to claim 29, wherein the electronic module further comprises a module memory connected to the module processor and operable to store a dosing history, the dosing history including a number of dose sizes sequentially dispensed or injected by the injection device and determined or calculated by the processor.

31. The add-on device according to claim 26, wherein the module processor is operable to read use-related data from the memory of the machine-readable identification and is further operable to determine or to calculate a residual amount of the medicament contained in the medicament container on the basis of the use-related data.

32. An injection system, comprising:
an injection device comprising:
a housing to accommodate a medicament container containing the medicament,
a drive mechanism for injecting of a dose of the medicament from the medicament container, and
a machine-readable identification comprising an electronic circuit with a writable memory configured to store at least one of a device information, a container information and a use-related data,
an add-on device comprising:
a device body detachably fastenable to a portion of the injection device,
an electronic module comprising a module processor and a transceiver operable to read, to receive, to alter or to generate at least one of the device information, the container information and the use-related data and wherein the electronic module is operable to store at least one of the device information, the container information and the use-related data in the memory.

33. The injection system according to claim 32, wherein the add-on device further comprises a sensor operable to quantitatively determine at least one of a position and a movement of a movable component of the drive mechanism relative to at least one of the housing, the medicament container and the device body when the add-on device is attached to the injection device and wherein the sensor is operable to generate a sensor signal being indicative of at least one of the position and the movement of the movable component.

34. The injection system according to claim 33, wherein the module processor is operable to determine or to calculate a size of the dose of the medicament, which is set or dispensed by the injection device, on the basis of the sensor signal.

35. The injection system according to claim 32, wherein the use-related data is indicative of a status of use of the injection device, which status of use is determinable by the add-on device.

36. A method of monitoring operation of an injection device configured for injecting of a dose of a medicament, the method comprising the steps of:
attaching an add-on device to the injection device, wherein the injection device comprises a machine-readable identification comprising an electronic circuit with a writable memory configured to store at least one of a device information, a container information and a use-related data,
at least one of receiving, reading, altering or generating at least one of the device information, the container information and the use-related data by an electronic module of the add-on device, and
storing at least one of the device information, the container information and the use-related data in the memory by the electronic module.

37. The method according to claim 36, wherein storing at least one of the device information, the container information and the use-related data includes establishing of a wireless communication link between a transceiver of the electronic module and an antenna of the electronic circuit of the machine-readable identification.
